(19) United States
(12) Patent Application Publication
(10) Pub. No.: US 2007/0213388 A1
(43) Pub. Date: Sep. 13, 2007

(54) Acetylcholinesterase Inhibitors and N-Methyl-D-Aspartate Antagonists Useful in the Treatment of of Cognitive Disorders

(76) Inventor: Gosse B. Bruinsma, Leiden (NL)

Correspondence Address: TRASK BRITT, P.O. BOX 2550, SALT LAKE CITY, UT, 84110, US

(21) Appl. No.: 10/593,215

(22) PCT Filed: Mar. 18, 2005
(86) PCT No.: PCT/US05/09186

§ 371 Date: Oct. 4, 2006

Related U.S. Application Data
(60) Provisional application No. 60/554,551, filed on Mar. 19, 2004.

(51) Int. Cl. A61K 31/407

(52) U.S. Cl. 514/411

(57) ABSTRACT

The present invention relates to compositions and methods of treating a cognitive disorder in a subject by administering an effective amount of phenserine or a pharmaceutically acceptable salt thereof and an effective amount of memantine or a pharmaceutically acceptable salt thereof to a subject, thereby treating a cognitive disorder in the subject.

### CROSS-REFERENCE TO RELATED APPLICATIONS

This application claims the benefit of U.S. Provisional Patent Application No. 60/554,551, filed Mar. 19, 2004, the entirety of which is hereby incorporated by reference.

### TECHNICAL FIELD

This invention relates to pharmaceutical compositions and methods of using a cholinesterase inhibitor, such as a phenserine compound, in combination with an N-Methyl-D-Aspartate agonist, such as memantine.

### BACKGROUND

Alzheimer's disease (“AD”) is a form of dementia in which subjects develop progressive neurodegeneration, complete loss of cognitive abilities, and die prematurely. Alzheimer's disease is a complex cognitive disorder associated with major structural changes in the brain; loss of neurons in the hippocampus and cortex, accumulation of intracellular protein deposits (neurofibrillary tangles) and accumulation of extracellular protein deposits (amyloid or senile plaques). The major component of the senile plaques is beta-amyloid peptide (Aβ) which resides within a much larger amyloid precursor protein (β-APP). Normal metabolism of β-APP involves proteolytic cleavage within the Aβ region which precludes the formation of amyloidogenic Aβ peptides, and favors the release of non-amyloidogenic soluble β-APP fragments into the extracellular fluid. Aβ formation can increase due to β-APP overexpression or missense mutations which alter constitutive β-APP processing pathways. Evidence suggests that although non-amyloidogenic β-APPs are neuroprotective, Aβ may be one of the main causes for cytotoxic processes leading to neuronal death in Alzheimer's disease.

In addition, defects in the cholinergic system have been suggested to underlie cognitive impairments associated with normal aging and Alzheimer's disease (Bartus et al., Science 217:408-417 (1982); Fisher et al., Neurobiol. Aging 13:9-23 (1992)). Much research has focused on the development of cholinomemetic replacement therapy as a potential treatment of these impairments. Among them, cholinesterase inhibitors, such as physostigmine (“Phy”) and tetrahydroaminoacridine (“ThA”) have been investigated for memory-enhancing effects in both animals (Rupniak et al., Neurobiol. Aging 11:09-613; 1990); Murray et al., Psychopharmacology 105:134-136 (1991) and human patients (Mohs et al., J. Am. Geriatr. Soc. 3:749-757 (1985); Summers et al., N. Engl. J. Med. 315:1241-1245(1986)).

Other agents have been proposed as selective inhibitors of acetylcholinesterase (“AChE”). Thus heptyl-physostigmine (“Heptyl-Phy”) was described as having greater lipophilicity, longer inhibitory action on cholinesterase and more persistent increases in acetylcholine in brain with less toxicity than the parent compound (Brufani et al., Pharmacol. Biochem. Behav. 26:625-629 (1987)). Concern exists, however, as to whether the therapeutic window of heptyl-Phy is wide enough for clinical use.

Phenserine ((−)-N-phenylcarbamoyl eseroline) has been identified as a superior, selective ACHE inhibitor (“AChEI”) and thus suited as an agent for the therapy for cognitive impairments associated with aging and Alzheimers disease (for example, see, U.S. Pat. Nos. 5,409,948; and 5,171,750, the contents of both of which are incorporated by this reference). The marked cholinergic loss in AD is accompanied by dramatic reductions in the enzymes cholineacetyl transferase, involved in the synthesis of the cholinergic neurotransmitter acetylcholine (ACh), and of AChE, which degrades ACh (Perry, et al. (1978) Brit. Med. J., 2 6150:1457-1459; Whitehouse, et al., (1982) Science 215:1237-1239.

Moreover, degeneration of neurons in Alzheimer's disease has been associated with a loss of glutamatergic synapses. Further, the glutamatergic system has been implicated in mediation of changes in β-APP metabolism, and as such Aβ formation may be enhanced by the deficits in glutamatergic neurotransmission and glutamate levels, as seen in the brains of Alzheimer's subjects.

At the present time Alzheimer's disease cannot be cured or prevented. However, strategies that reduce β-APPs production, prevent the formation of Aβ, or reduce Aβ toxicity, may retard the progression of Alzheimer's disease.

### SUMMARY OF THE INVENTION

The invention relates to a method of treating and/or delaying the onset or progression of a cognitive disorder in a subject, the method comprising administering an effective amount of a phenserine compound or a pharmaceutically acceptable salt or ester thereof to a subject and administering an effective amount of memantine or a pharmaceutically acceptable salt thereof to the subject, thereby treating and/or delaying the onset or progression of the cognitive disorder in the subject.

The present invention also relates to a method of treating and/or delaying the onset or progression of a cognitive disorder by administering an effective amount of memantine, which is between about 10 mg and about 20 mg per day, and an effective amount of a phenserine compound, which is between about 5 mg and about 100 mg per day.

The present invention further relates to a method of treating and/or delaying the onset or progression of, and a composition useful in the treatment or delay of, cognitive disorders, for example, AD, dementia, age related dementia, vascular dementia, Aβ neurotoxicity and/or Parkinson's disease. In an exemplary embodiment, an AChEI and an NMDA antagonist are coadministered to a subject to improve cognitive function and retard progression of the cognitive disorder, for example, AD.

The present invention also relates to a method of manufacturing a pharmaceutical composition comprising a phenserine compound or a pharmaceutically acceptable salt thereof and memantine or a pharmaceutically acceptable salt thereof for the treatment of AD, age related dementia, dementia, vascular dementia, and/or Parkinson's disease.

### DETAILED DESCRIPTION OF THE INVENTION

AD is a degenerative condition affecting memory, judgment and the ability to reason that affects about 4.5 million Americans. The present invention provides an improved method of treating cognitive diseases, such as AD, dementia, vascular dementia, and Parkinson's disease.

Drugs, such as phenserine are thought to act by increasing the availability of the neurotransmitter acetylcholine. In contrast, N-methyl-D-aspartate (“NMDA”) antagonists are thought to work by blocking the action of glutamate. The present invention provides a beneficial use of an NMDA antagonist in combination with a phenserine compound to produce an improved treatment. For example, co-administration of an NMDA antagonist, which can prevent glutamate toxicity and reduce production or accumulation of Aβ, and a phenserine compound, which is an AChEI and/or reduces production of β-APP, provides a treatment which may help prevent neuronal cell death and/or provides additional neurotransmitter availability and/or decreases cholinergic over stimulation, thereby boosting the cognitive ability of a subject and/or slowing progression of the disease. In an exemplary embodiment, the NMDA antagonist does not reduce cognitive abilities in a subject, i.e., leaning or memory (Longo, P. M. and S. M. Massa, (2004) Neuroprotective Strategies in Alzheimer's Disease, NeuroRx® 1:117-127, incorporated by reference).

As used herein, the phrases “co-administration,” “in combination with,” “the combination of” or similar phrases referring to two or more drugs or compounds means that the compounds are present in the subject being treated at the same time. The compounds may be administered at the same time or sequentially in any order at different points in time. However, the compounds should be administered sufficiently closely in tune so as to provide the desired enhancement of treatment effect. The compounds may be administered by the same route of administration or by different routes of administration. Suitable dosing intervals, routes and the order of administration with such compounds will be readily apparent to those skilled in the art, in light of the present disclosure.

As used herein, “treating” or “treatment” does not require a complete cure. It means that the symptoms of the underlying disease or associated conditions are at least reduced and/or delayed, and/or that one or more of the underlying cellular, physiological, or biochemical causes or mechanisms causing the symptoms are reduced, delayed and/or eliminated. It is understood that reduced or delayed, as used in this context, means relative to the state of the untreated disease, including the molecular state of the untreated disease, not just the physiological state of the untreated disease.

As used herein, “effective amount” means an amount of an active ingredient administered to the subject, which will be effective to improve or treat the disease condition in the subject.

As used herein, a “phenserine compound” means phenserine and/or (+)-phenserine and pharmaceutically acceptable salts and esters thereof. As used herein, “phenserine” means the negative enantiomer, (−)-phenserine, and pharmaceutically acceptable salts and esters thereof. Reference to a compound, such as a phenserine compound, phenserine and/or (+)-phenserine shall be understood to include reference to pharmaceutically acceptable salts and esters of the compound or compounds.

Pharmaceutically acceptable salts include tartrate, formate, citrate, salicylate, fumerate, oxalate, phosphate, succinate, maleate, phenylsuccinate, hydrochloride, hydrobromide, sulfonate, benzenesulfonate, naphthalenesulfonate, hydroidate, sulfamate, sulfate, acetate, trifluoroacetate, trichloroacetate, gluconate, benzoate, lactate, methanesulfonate, ethanesulfonate, benzenesulfonate, choline hydrochlorate, p-toluenesulfonate, cyclolexylsulfonate, cyclohexylsulfamate, quinate, bicarbonate, bisulfate, bitartrate, borate, bromide, calcium edetate, camsylate, carbonate, chloride, clavulanate, dihydrochloride, edetate, edisylate, estolate, esylate, gluceptate, gluconate, glutamate, glycollylarsanilate, hexylresorcinate, hydrabamine, hydroxynaphthoate, iodide, isethionate, lactobionate, laurate, malate, mandelate, mesylate, methylbromide, methylnitrate, methylsulfate, monopotassium maleate, mucate, nitrate, N-methylglucamine, glucoheptonate, laurylsulphonate, pamoate (embonate), palmitate, pantothenate, diphosphate, polygalactumate, potassium, sodium, stearate, subacetate, tannate, teoclate, triethiodide, trimethylammonium, oleate and/or valerate.

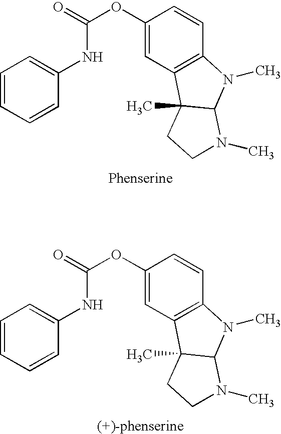

The NMDA receptor requires simultaneous binding by glutamate and glycine to activate the ion channel (i.e., to open the channel). In addition to the binding sites for glutamate and glycine, the NMDA receptor contains other distinct modulatory sites to which Me²⁺, Zn²⁺, polyamines, and exogenous ligands bind. The binding sites for Mg²⁺ and exogenous ligands, such as memantine, which is a NMDA antagonist, are located within the channel and are accessible for pharmacologic modulation only when the channel is activated or in the “open state.” Therefore, memantine may be referred to as an “open channel,” “uncompetitive” or “use-dependent” NMDA receptor antagonist.

Memantine has been reported to help treat the symptoms of AD in some subjects, but it has not been reported to modify the underlying pathology of the disease. Memantine (as the hydrochloride salt) is 1-amino-3,5-dimethyladamantane hydrochloride, with a molecular formula of C₁₂H₂₁N•HCl (1-amino-3,5-dimethyladamantine-hydrochloride). Description of memantine shall be understood to include the active and less toxic metabolite, 1-amino-3-hydroxymethyl-5-methyl adamantane (Mrz 2/373) and pharmaceutically acceptable salts and esters thereof. The IC₅₀ of memantine on neuronal receptors, at 67 mV, is 1.4 μM. It is commercially available from Merz & Co., Frankfurt, Germany.

Memantine is a low to moderate affinity, uncompetitive N-methyl-D-aspartate, voltage dependent, receptor antagonist with rapid blocking and unblocking kinetics. Thus, memantine is thought to block sustained activation of the receptor by glutamate under pathological conditions (excitotoxicity), and to rapidly leave the NMDA receptor channel during normal physiological activation. These features also distinguish memantine from other NMDA receptor antagonists (e.g., dissociative anesthetics, ketamine, and MK-801) and confer good safety and tolerability with a relatively high therapeutic margin. Thus, memantine administration is believed to decrease the neuronal toxicity and neurodegeneration associated with excessive glutamate release. Furthermore, memantine is not believed to impair learning or memory. In contrast, other NMDA antagonists typically inhibit learning and long term potentiation (“LTP”) (see Kelly et al. (1997) Response-reinforcement learning is dependent on NMDA receptor activation in the nucleus accumbens core, Proc. Natl. Acad. Sci. USA 94:12174-12179).

In humans, memantine is bioavailable after an oral dose, undergoes minimal metabolism, excreted primarily in the urine, and exhibits a terminal elimination half-life of 60 to 80 hours. Memantine is water soluble and rapidly crosses the blood brain barrier with a CSF/serum ratio of 0.52. Memantine does not inhibit cytochrome P450 (CYP 450) isoenzymes in vitro, and the pharmacokinetics are reportedly not affected by food, sex, or age.

Dosages of memantine may be determined by a physician, for instance, memantine in a dose of up to 20 mg/day, may be used to treat AD. For example, memantine may be administered at a dosage of 10 mg/day. Currently, memantine is administered at a dosage of about 10 mg twice a day. The present invention contemplates the use of memantine at dosages between about 1 mg and about 40 mg, including any whole number therebetween, such as 5 mg, and such dosages may be administered at more than one time per day, for example, once or twice a day. Memantine has exhibited an acceptable safety and tolerability profile in a variety of neurodegenerative disorders (e.g., dementia, neuropathic pain, spasticity, and Parkinson's disease).

Phenserine is a highly selective AChEI that is less toxic, compared to physostigmine and tacrine, and robustly enhances cognition in animal models (see, Greig N H et al. (2000) The Experimental Alzheimer Drug Phenserine: Preclinical Pharmacokinetics and Pharmacodynamics, Acta Neurol Scand Suppl. 176:74-84). To determine the time-dependent effects of phenserine on cholinergic function, AChE activity, brain and plasma drug levels and brain extracellular acetylcholine (ACh) concentrations were measured in rats before and after phenserine administration. Following i.v. dosing, brain drug levels were 10-fold higher than those achieved in plasma, peaked within 5 min and rapidly declined with half-lives of 8.5 and 12.6 min, respectively. In contrast, a high (>70%) and long-lasting inhibition of AChE was achieved (half-life >8.25 h). Striatal, in vivo microdialysis in conscious, freely-moving phenserine-treated rats demonstrated a greater than 3-fold rise in brain ACh levels. Phenserine, thus, is rapidly absorbed and cleared from the body, but produces a long-lasting stimulation of brain cholinergic function at well tolerated doses and hence has superior properties as a drug for cognitive disorders, such as AD. Further, the selective inhibition of AChE minimizes the potential BChE side effects. Its long duration of action, coupled with its short pharmacokinetic half-life, reduces dosing frequency, decreases body drug exposure and minimizes the dependence of drug action on the individual variations of drug metabolism commonly found in the elderly. Thus, a person of ordinary skill in the art would not combine a cognitive ability increasing drug like phenserine with a typical NMDA antagonist, which would be expected to negatively affect the function of phenserine.

Cholinergic stimulation, for example, by administration of an AChE inhibitor like phenserine reduces glutamate release (Colgin et al. (2003) Septal Modulation of Excitatory Transmission in Hippocampus, J. Neurophysiol. 90:2358-2366; Colgin et al. (2003) Cholinergic plasticity in the Hippocampus, Proc. Natl. Acad. Sci. USA 100(5):2872-2877). Therefore, a lower dose of memantine may be used to effectively treat a cognitive disease when memantine is coadministration with phenserine, thereby decreasing undesirable side effects. In addition, coadministration of a phenserine compound and memantine may provide a synergistic effect by both reducing glutamatergic stimulation through the action of phenserine and/or reducing or preventing glutamate induced neuronal toxicity through the action of memantine and the Aβ effects of phenserine and/or isomers thereof, e.g., the (+) enantiomer of phenserine.

Stojiljkovic et al. “Memantine Treatment Improves Antidotal Efficacy Of Atropine, Hi-6 And Diazepam In Rats Poisoned With Soman” reports that memantine and its metabolite (Mrz 2/373) along with atropine, HI-6 and diazepam were better than physostigmine or pyridostigmine in providing prophylaxis in soman-intoxicated rats. Co-administration of HI-6 and Mrz 2/373 only produced a significant difference in erythrocyte acetylcholinesterase activity from the soman alone group, the authors concluded that the results differed from those previously cited. This report did not address a phenserine compound and memantine for the treatment of a cognitive disease and found that their results were not consistent with prior results.

In an exemplary embodiment, memantine is coadministered to a subject having a cognitive disease or thought to be at risk of developing a cognitive disease with phenserine. Without wishing to be bound by theory, coadministration of phenserine and memantine is believed to provide multiple separate mechanisms of neuronal protection, reduced accumulation of Aβ and reduction in glutamate induced toxicity, and cognitive improvement by way of cholinergic stimulation. Hence, the treatment of the present invention provides a significant improvement in the art by both treating the disease and preventing or slowing progression of the disease.

In another exemplary embodiment, memantine is coadministered to a subject having a cognitive disease or thought to be at risk of developing a cognitive disease with both phenserine and (+)-phenserine. Without wishing to be bound by theory, it is believed that the combination of phenserine and (+)-phenserine further reduces synthesis of β-APP (and accumulation of Aβ) without producing cholinergic over stimulation. Hence, the dosage of (+)-phenserine, which lacks cholinesterase activity (see, WO 00/248150), in the present embodiment may be adjusted by a person of ordinary skill in the art using the guidance of the present invention to effectively reduce the production of Aβ without producing cholinergic overstimulation. Thus, the relative amounts of phenserine and (+)-phenserine administered to a subject may be adjusted to achieve the desired level of cholinergic stimulation and β-APP repression, for example, if a dosage of 15 mg bid of phenserine and 30 mg bid of (+)-phenserine is found to produce a desirable level of β-APP inhibition, but increased cholinergic stimulation is desired, the dosage of phenserine may be increased, for example, to 20 or 30 mg bid and the dosage of (+)-phenserine decreased to 25 or 15 mg bid, respectively.

In another exemplary embodiment, memantine is coadministered with (+)-phenserine to a subject having a cognitive disease or thought to be at nsk of developing a cognitive disease. Without wishing to be bound by theory, and coadministration of (+)-phenserine and memantine is believed to provide two separate mechanisms of neuronal protection, reduced accumulation of Aβ and reduction in glutamate induced toxicity. Hence, the treatment of the present invention provides a significant improvement in the art by preventing the onset or slowing progression of a cognitive disease.

The effective dose of phenserine for mammals, for example, a human, may vary due to such factors as age, weight, activity level or condition of the subject being treated. Typically, an effective dosage of a compound according to the present invention is about 1 to 800 milligrams when administrated by either oral or rectal dose from 1 to 3 times daily. This dosage is typically about 0.002 to about 50 milligrams per kilogram of the subject's weight administered per day. Preferably, from about 10 to about 300 milligrams are administered orally or rectally 1 to 3 times a day for an adult human. The required dose is considerably less when administered parenterally. Preferably, from about 0.01 to about 150 milligrams may be administered intramuscularly or transdermally, one or two times a day for an adult human.

Phenserine and (+)-phenserine, which are both phenserine compounds, can be administered in any pharmaceutically acceptable amount, for example, in amounts ranging from 0.001 gram to about 1 gram per kilogram of body weight. In an exemplary embodiment, the compound is administered in a dosage of between about 5 mg and about 120 mg, including 5 mg twice a day (bid), 10 mg bid, 15 mg bid, 20 mg bid, 25 mg bid, 30 mg bid, 35 mg bid, 40 mg bid, 45 mg bid, 50 mg bid, 55 mg bid, 60 mg bid. In another exemplary embodiment, the compound is administered in a dosage of 7.5 mg twice a day, or 15 mg twice a day. In yet another exemplary embodiment, the compound is administered in a dosage of 10 mg twice a day. However, based on the information which is presented herein, the determination of additional effective amounts is well within the skill of the ordinary practitioner in the art.

The oral administration/ingestion of phenserine and other cholinesterase inhibitors elicits a lesser response in a subject, as compared to an equal dosage administrated parenterally, due to metabolism of the drug during transit through the gastrointestinal tract and into the general circulation system. Thus, the metabolic breakdown of the active drug may be at least partially circumvented by administering the drug by an alternative route. Examples of such alternative routes include buccal or sublingual administration and parenteral administration. Drugs administered by these routes avoid gut-wall and hepatic metabolism, thereby producing increased bioavailability as compared to oral administration. Neither buccal nor sublingual administration of phenserine is known from the prior publications or patents, nor is a beneficial sustained release plasma profile found in prior publications or patents.

The compounds of the invention are generally used in pharmaceutical compositions (wt %) containing the active ingredient with a carrier or vehicle in the composition in an amount of about 0.1 to 99 wt % and preferably about 25-85 wt %. The compounds may be formulated for pharmaceutical use using methods known in the art. See, for example, Remington's Pharmaceutical Sciences, 18th Ed. (1990, Mack Publishing Co., Easton, Pa.). Accordingly, incorporation of the active compounds and a slow release matrix may be implemented.

Either fluid or solid unit dosage forms can be readily prepared for oral administration. For example, admixed with conventional ingredients such as dicalcium phosphate, magnesium aluminum silicate, magnesium stearate, calcium sulfate, starch, talc, lactose, acacia, methyl cellulose and functionally similar materials as pharmaceutical excipients or carriers. A sustained release formulation may optionally be used. In older or incoherent subjects sustained release formulations may even be preferred. Capsules may be formulated by mixing the compound with a pharmaceutical diluent which is inert and inserting this mixture into a hard gelatin capsule having the appropriate size. If soft capsules are desired, a slurry of the compound with an acceptable vegetable, light petroleum or other inert oil can be encapsulated by forming into a gelatin capsule.

Suspensions, syrups and elixirs may be used for oral administration or fluid unit dosage forms. A fluid preparation including oil may be used for oil soluble forms. A vegetable oil such as corn oil, peanut oil or a flower oil, for example, together with flavoring agents, sweeteners and any preservatives produces an acceptable fluid preparation. A surfactant may be added to water to form a syrup for fluid unit dosages. Hydro-alcoholic pharmaceutical preparations may be used having an acceptable sweetener, such as sugar, saccharin or a biological sweetener and a flavoring agent in the form of an elixir.

Pharmaceutical compositions for parenteral and suppository administration can also be obtained using techniques standard in the art.

In an exemplary embodiment, the compounds of the invention are prepared as pharmaceutical agent suitable for oral administration. In another exemplary embodiment, the compounds of the invention are prepared in transdermal parenteral formulation, which is particularly useful in preventing or treating cholinergic disorders such as AD. Accordingly, compositions suitable for administration to these areas are particularly included within the invention. The above parenteral solutions or suspensions may be administered transdermally with a skin patch. In addition, where appropriate and desirable they may be given by injection in an appropriate vehicle, such as sesame oil.

Pharmaceutical carriers acceptable for the purposes of this invention are the known art carriers that do not adversely affect the drug, the host, or the material comprising the drug delivery device. Suitable pharmaceutical carriers include sterile water, saline, dextrose, dextrose in water or saline, condensation products of castor oil and ethylene oxide combining about 30 to 35 moles of ethylene oxide per mole of castor oil, liquid acid, lower alkanols, oils such as corn oil, peanut oil, sesame oil and the like, with emulsifiers such as mono- or di-glyceride of a fatty acid; or a phosphatide, e.g., lecithin, and the like; glycols, polyalkylene glycols, aqueous media in the presence of a suspending agent, for example, sodium carboxymethyl cellulose, sodium alginate, poly(vinylpyrrolidone), and the like, alone or with suitable dispensing agents such as lecithin, polyoxyethylene stearate, and the like. The carrier may also contain adjutants such as preserving agents, stabilizing agents, wetting agents, emulsifying agents and the like together with the compounds of this invention.

Suitable salts of the compounds of the invention, such as acid addition salts, which may be prepared according to a conventional procedure, include the following acids: hydrochloric, hydrobromic, methanesulfonic, isothionic, sulfuric, phosphoric, and sulfamic acids and, from the organic series: acetic, propionic, maleic, fumaric, tartaric, citric, oxalic, and benzoic acids, to name a few. Memantine acids include, hydrochloric, citric, and maleic. Other pharmaceutically-acceptable acid addition salts may be prepared, if desired, and one acid addition salt may be converted into another by neutralizing one salt, for example, the hydrochloride, resulting in the free base, and then reacidiying with a different selected mineral or organic acid, to prepare another pharmaceutically-acceptable acid addition salt, as is conventional in the art.

A variety of measures to evaluate the effect of memantine and/or phenserine on the cognitive ability of a subject are known in the art. For example, the Severe Impairment Battery (SIB) to assess attention, orientation, language, memory, and social interactions, the modified AD Cooperative Study—Activities of Daily Living (ADCS-ADL) scale, which assesses the ability of subjects to eat, dress, bathe, travel shop and perform household chores, the Behavioral Rating Scale for Geriatric subjects (BGP), which assesses day-to-day functioning, and the Clinical Global Impression of Change (CGI-C), which assesses the overall condition of the subjects, may be used. The measure of cognitive ability may be used to monitor the progression of the disease, relative to untreated subjects.

The cognitive effect may be assayed using a T-maze (Patel et al., (1998) Phenserine, a Novel Acetylcholinesterase Inhibitor, Attenuates Impaired Learning of Rats in a 14-unit T-maze Induced by Blockade of the N-methyl-D-aspartate receptor, NeuroReport 9(1):171-176). In addition, Pavlovian fear conditioning may be used to assay cognitive function. For example, mice may be condition by receiving 3 to 5 tone-foot shock trials in a conditioning chamber (see, Maren, S. (1999) Neurotoxic Basolateral Amygdala Lesions Impair Learning and Memory But Not the Performance of Conditional Fear in Rats, J. Neurosci. 19(19):8696-8703).

For total Aβ levels, the rabbit polyclonal antibody no. 3160 (1-40 residues of Aβ is used as a capture antibody for all species of Aβ (Aβ1-40 and Aβ1-42), whereas mAb 4G8 (17-25 residues of Aβ) is used to detect Aβ levels, and the values are expressed as the mean of independent assays (see, Suzuki, N., et al. (1994) Science 264:1336-1340).

Total Aβ and Aβ42 levels may also be assayed in guanidine lysates as described (Johnson-Wood, et al. (1997) Proc. Natl. Acad. Sci. USA 94:1550-1555.). In brief tissue, e.g., hippocampal or cortical, is homogenized in a denaturing buffer containing 5 M guanidine plus protease inhibitors. The extracts are diluted and analyzed in denaturing ELISAs containing a final concentration of 500 mM guanidine for total A0 or Aβ42.

To evaluate the potential soluble pools of brain Aβ, a carbonate extraction may be performed (100 mM carbonate/50 mM NaCl/protease inhibitors, pH 11.5), for example, on hippocampal and cortical tissue (1:20, wt/vol) on ice. Tissue samples are Dounce homogenized and spun in a microcentrifuge at 14,000 rpm for 0.15 min at 4° C. The supernatant is placed in a fresh tube on ice and the pH of the lysate is neutralized to 7.4 with 1 M Tris (pH 6.8). The carbonate soluble pool of total Aβ is determined with denaturing (guanidine) and nondenaturing (lacking guanidine) ELISAs. An additional Aβ ELISA may be used to identify possible oligomeric species of Aβ. A monoclonal antibody directed against the first five residues of Aβ is used for both capturing and detecting Aβ.

Co-administration of an NMDA antagonist, such as memantine, and an AChEI, such as a phenserine compound, provides an improved treatment for cognitive disorders. For example, the dosage of phenserine may be effectively limited by the response of a subject's cholinergic system. Over stimulation of the cholinergic system, which may result from high doses of AChEIs like phenserine, can produce trembling and other undesirable side effects. The method of the present invention allows the dose of the AChEI to be adjusted to provide a desirable level of cholinergic treatment. This cholinergic effect is augmented by co-administration of a NMDA antagonist, such as memantine, which prevents over stimulation of NMDA receptors and excitotoxicity and does not adversely interact with AChEIs. Further, both a phenserine compound and memantine reduce Aβ accumulation and thereby reduce the toxic effect of this peptide. Therefore, co-administration of phenserine and memantine provides an improved treatment for cognitive disorders.

The invention is further explained with the aid of the following illustrative Examples.

### EXAMPLE I

To evaluate the role of memantine and/or phenserine in AD pathology, PDAPP homozygous mice are used to asses the effect of the drugs. Control animals receiving neither drug are evaluated against animals receiving one or both drugs. For example, memantine is administered at a dosage of 5 or 10 mg/kg and/or phenserine is administered at a dosage of 1 or 2 mg/kg.

Four treatment groups are established having at least 6 animals per group, wherein the animals are old (12-14 month) PDAPP mice homozygous (+/+) for the APP V717F transgene, a transgenic mouse model that develops AD-like neuropathology. Treatment group 1 is a control group receiving vehicle only, group 2 receives memantine at 5 mg/kg bid, group. 3 receives phenserine at 5 mg/kg bid, and group 4 receives both memantine and phenserine at 5 mg/kg bid. Animals are treated for 4 weeks, with cognitive ability tested prior to treatment and at appropriate times throughout the treatment period.

Animals receiving both phenserine and memantine are found to have improved cognitive function and to show reduced AD levels, for example, Aβ levels are reduced relative to both the control group and groups 2 and 3.

### EXAMPLE II

Capsules containing 10 mg of memantine and 15 mg of phenserine are made by incorporating pharmaceutically acceptable salts of memantine and phenserine into a gelatin capsule.

### EXAMPLE III

To evaluate the role of memantine and/or phenserine and/or (+)-phenserine in AD pathology, PDAPP homozygous mice are used. Control animals receiving vehicle only are evaluated against various test groups receiving: memantine (0.1 mg/kg); memantine (1 mg/kg); memantine (2 mg/kg); (+)-phenserine (0.5 mg/kg); phenserine (0.5 mg/kg); (+)-phenserine (1 mg/kg); phenserine (1 mg/kg); memantine (2 mg/kg) and (+)-phenserine (1 mg/kg); memantine (2 mg/kg) and phenserine (1 mg/kg); memantine (2 mg/kg), (+)-phenserine (1 mg/kg) and phenserine (1 mg/kg); memantine (2 mg/kg); (+)-phenserine (1 mg/kg); phenserine (1 mg/kg); (+)-phenserine (2 mg/kg); phenserine (2 mg/kg); memantine (2 mg/kg) and (+)-phenserine (1 mg/kg); memantine (2 mg/kg) and phenserine (1 mg/kg); memantine (2 mg/kg), (+)-phenserine (1 mg/kg) and phenserine (1 mg/kg); memantine (2 mg/kg) and (+)-phenserine (2 mg/kg); memantine (2 mg/kg) and phenserine (2 mg/kg); and memantine (2 mg/kg), (+)-phenserine (2 mg/kg) and phenserine (2 mg/kg). As will be recognized by a person of ordinary skill in the art, the dosages of each drug may be altered so as to assist in the identification of synergistic interaction between memantine and a phenserine compound, e.g., phenserine.

Each treatment group is established having at least 6 animals per group, wherein the animals are old (12-14 month) PDAPP mice homozygous (+/+) for the APP V717F transgene, a transgenic mouse model that develops AD-like neuropathology. Animals are treated for 4 weeks, with cognitive ability tested prior to treatment and at appropriate times throughout the treatment period.

Animals receiving both phenserine and memantine are found to have improved cognitive function and/or to show reduced Aβ levels, relative to both the control groups and to animals receiving a sub-optimal dosage of either drug alone.

All references, including publications, patents, and patent applications, cited herein are hereby incorporated by reference to the same extent as if each reference were individually and specifically indicated to be incorporated by reference and were set forth in its entirety herein.

1. A method of treating a cognitive disorder in a subject, said method comprising:
administering an effective amount of a first compound comprising a phenserine compound to a subject; and
administering an effective amount of a second compound comprising memantine to the subject, thereby treating a cognitive disorder in said subject.

2. The method according to claim 1, wherein said effective amount of the second compound is between about 10 mg and about 20 mg per day and said effective amount of the first compound is between about 10 mg and about 60 mg per day.

3. The method according to claim 2, wherein said effective amount of second compound is about 10 mg/day.

4. The method according to claim 2, wherein said first compound is phenserine and said effective amount of first compound is about 15 mg twice a day.

5. The method according to claim 4, wherein said effective amount of second compound is about 10 mg/day twice a day.

6. The method according to claim 1, wherein said first compound is (+)-phenserine and said effective amount of first compound is between about 5 mg and about 100 mg.

7. The method according to claim 1, wherein the first compound comprises both (+)-phenserine and phenserine.

8. The method according to claim 7, wherein the first compound comprises (+)-phenserine tartrate and phenserine tartrate.

9. The method according to claim 1, wherein said cognitive disorder is selected from the group consisting of Alzheimer's disease, vascular dementia, Parkinson's disease, and combinations of any thereof.

10. The method according to claim 9, wherein said cognitive disorder is Alzheimer's disease.

11. The method according to claim 1, wherein administering said effective amount of said first compound and said effective amount of second compound comprises administering a pharmaceutically acceptable composition comprising phenserine and memantine.

12. A method for reducing neurotoxicity of beta-amyloid protein in a subject receiving memantine comprising administering a phenserine compound to the subject.

13. The method according to claim 12, wherein the phenserine compound is phenserine.

14. The method according to claim 12, wherein the phenserine compound is (+)-phenserine.

15. The method according to claim 12, wherein the phenserine compound comprises a tartrate salt.

16. A composition comprising an effective amount of a first compound comprising a phenserine compound and an effective amount of a second compound comprising memantine, wherein said composition is useful in the treatment of a cognitive disorder.

17. The composition of claim 16, comprising one or more pharmaceutically-acceptable diluents, excipients, or carriers.

18. The composition of claim 17, wherein said effective amount of the first compound is 15 mg.

19. The composition of claim 17, wherein said effective amount of the second compound is 10 mg.

20. The composition of claim 16, wherein said effective amount of the second compound is between about 5 mg and about 20 mg.

21. The composition of claim 16, wherein said effective amount of the first compound is between about 10 mg and about 60 mg.

22. The composition of claim 16, wherein said effective amount of the second compound is between about 10 mg and about 20 mg and said effective amount of the first compound is between about 1 mg and about 30 mg.

23. The composition of claim 17, wherein said cognitive disorder is Alzheimer's disease.

24. The composition of claim 16, wherein the first compound comprises a tartrate salt of a phenserine compound.

25. The composition of claim 16, wherein the second compound comprises a pharmaceutically acceptable salt of memantine.

26.-40. (canceled)
